(19) United States
(12) Patent Application Publication
(10) Pub. No.: US 2008/0206869 A1
(43) Pub. Date: Aug. 28, 2008

(54) Nucleic Acid Complex

(75) Inventors: Edvard Smith, Stockholm (SE); Mathias Svahn, Stockholm (SE); Oscar Simonson, Stockholm (SE)

Correspondence Address: YOUNG & THOMPSON, 209 Madison Street, Suite 500, ALEXANDRIA, VA, 22314, US

(73) Assignee: AVARIS AB, STOCKHOLM (SE)

(21) Appl. No.: 11/814,646

(22) PCT Filed: Jan. 23, 2006
(86) PCT No.: PCT/SE06/00092

§ 371 Date: Jul. 24, 2007

Related U.S. Application Data
(60) Provisional application No. 60/645,752, filed on Jan. 24, 2005.

(51) Int. Cl. C12N 15/00; C07H 21/02; C12N 5/00

(52) U.S. Cl. 435/440; 536/24.5; 435/325

(57) ABSTRACT

The present invention relates to modification of nucleic acids for specific delivery in vitro and in vivo. More specifically, the present invention relates to modification of RNA or DNA molecules in order to add functions in terms of delivery and specificity to RNA interference or antisense technology. A specific binding domain is incorporated into the nucleic acid to which a complementary nucleic acid, conjugated to a biologically active molecule, can hybridize.

### FIELD OF THE INVENTION

The present invention relates to modification of nucleic acids and in particular modification of short interfering and short hairpin RNAs and oligonucleotides possessing antisense activity for specific delivery in vitro and in vivo.

### BACKGROUND OF THE INVENTION

RNA interference (RNAi) is a phenomenon in which the gene expression is suppressed by a process triggered by double-stranded RNA (dsRNA) homologous to the silenced gene. RNAi is a natural mechanism, which can also be used to provide information about gene function and has become a useful research tool for many organisms. The use of RNAi for genetic-based therapies is widely studied, especially in viral infections, cancers, and inherited genetic disorders.

Inhibition is caused by the specific degradation of the mRNA transcribed from the target gene. dsRNA is processed by cleavage into shorter units (called short interfering RNA; siRNA) that guide recognition and targeted cleavage of homologous target mRNA.

Typically 21mers of dsRNA, with 2 nucleotides overhang on each 3′-end, has shown to be able to down regulate protein production by reducing the RNA level. The mechanism is not fully understood nor the strategy of design. General rules for the design have however been presented along with chemical modifications of both strands for increased silencing.

siRNA can be introduced to the cell either as synthetic siRNA or as a plasmid expressing longer double stranded RNA, later cleaved by Dicer, an RNase III-family enzyme (step 1 in FIG. 1). The RISC (RNA-Induced Silencing Complex) complex preferably binds to the strand with the lowest ΔG (Gibbs' free energy) at the 5′-end. The siRNA/RISC complex subsequently localizes the target RNA, and the antisense strand hybridizes to it. Upon hybridization the siRNA/RNA structure is recognized by the cell and the targeted RNA is cleaved.

siRNA is also capable of repressing gene expression at the transcriptional level.

The mechanism is not completely understood but it has been shown that siRNA targeted to CpG islands of a promoter can induce DNA and histone methylation in cells. The same type of design of siRNAs can be used for transcriptional gene silencing and for post-transcriptional silencing, Kawasaki H, et al. Nature. 2004 September 9; 431(7005):211-217

As mentioned previously, siRNA can be introduced to the cell either as synthetic siRNA or as a plasmid expressing longer double stranded RNA. When starting with synthetic siRNA no cleavage by the Dicer complex is needed. The smallest molecule to start with is to synthesize typically 21mers of double stranded RNA with 2 nucleotides overhang at each 3′-end. Even though digestion by Dicer is thought to launch the siRNA into the RISC and thus, increase RNAi efficacy, small molecules have a better chance of entering a cell in vivo. Utilizing biologically active molecules as ligands to generate entrance through receptor-mediated endocytosis is generally easier with a small complex, especially if only a few biologically active molecules are used.

siRNA is not readily taken up into cells. Complexes that increase transfer within an organism outside the target cell, across the cellular membrane and/or within the target cell would greatly enhance the usage of this novel technology.

WO 00/15824 discloses a method and complex, Bioplex, for transfer of a nucleic acid across a biological membrane and specific localization of said nucleic acid within a cell. The method is based on the use of a synthetic transport entity composed of a functional element (herein also referred to as FE and functional entity) and a binding element (herein referred to as anchor). The functional element can be, for example, a Nuclear Localization Signal (NLS) that confers a specific biological function to a molecule linked to it. In contact with the biological membrane the transport entity will provide for a transfer of the nucleic acid of interest across the biological membrane.

WO 03/091443 further improves the Bioplex technology of WO 00/15824 with respect to safety, functionality, efficiency and stability. The transport entity disclosed in WO 03/091443 can be altered in a controlled manner prior to the use in a biological system, at the surface of the membrane or after having passed across the membrane or having been taken up by the cell, and comprises at least one alteration site that, when altered, changes a property of the transport entity. WO 03/091443 also relates to a method for transfer the transport entity across a biological membrane, and/or direction thereof to a specific location within a cell.

Several attempts to modify siRNA have been suggested in order to alter or improve their effectiveness. Enhancement of RNAi activity by improved siRNA duplexes was described by Hohjoh in FEBS Letters 557(2004) pp. 193-198. Various siRNA duplexes were constructed against the P. luciferase gene and the effect of the duplexes on the suppression of the expression of P. luciferase was examined by cotransfection of the duplexes with a pGL3-control plasmid carrying the P. luciferase gene and a phRL-TK-plasmid carrying the R. luciferase gene as a control into HeLa cells. One to four mismatches introduced at the 3′-end of the sense strand of a siRNA duplex were shown to enhance RNAi activity over conventional siRNA duplexes in cultured mammalian cells.

Similarly, WO 03/064621 describes that one or more mismatches can be introduced along the length of a siRNA duplex. The mismatched bases should be in the sense strand of the siRNA in order not to reduce the binding affinity of the anti-sense strand for the mRNA target. According to description of WO 03/064621, the siRNAs produced by the disclosed methods were significantly more potent than previously available siRNAs.

Soutschek et al., Nature, vol 432 (2004) pp. 173-178 presented conjugation of cholesterol to the 3′ end of the sense strand of a siRNA molecule by means of a pyrrolidine linker, thereby generating a covalent and irreversible conjugate. This chemically modified siRNA resulted in silencing of the apoB mRNA in liver and jejunum, decreased plasma levels of apoB protein and reduced total cholesterol. According to Soutschek et al. the modification did not result in a significant loss of gene-silencing activity in cell culture.

Cholesterol will give a rather unspecific tissue uptake. On the other hand, since it is an endogenous substance, there will be no risk of eliciting an immune response. Large molecules, such as proteins, are known to elicit immune responses especially if they derive from a microorganism. The possibility of readministration has to be considered. Another drawback of having a larger molecule chemically conjugated to the siRNA is that it might shield the siRNA from RISC.

Additions of proteins and other biologically active molecules, by chemical linking to the siRNA, would require a lot of effort. Some endosomal pathways rely on multiple receptor-ligand interactions. Combinatorial ligand uptake studies with siRNAs with covalent chemistry would be tedious and very inflexible. More than one biologically active molecule might be needed since there are multiple thresholds for an efficient delivery of siRNA. Specific activities are needed for specific tasks in the routing of the delivery.

Covalent conjugation is also very inflexible in the choice of siRNA sequence. A mix of siRNAs targeting the same RNA is sometimes more potent than a single siRNA. Subsequently, covalent conjugation needs to be performed for every siRNA sequence. If the same biologically active molecule is to be used in another setup, i.e. a new set of siRNAs targeting another RNA, all siRNAs have to be conjugated separately.

The present invention avoids these problems by utilizing and modifying the plasmid based technology described in WO 00/15824 and WO 03/091443 and adds functions to siRNA/short hairpin RNA molecules after conventional RNA synthesis.

### SUMMARY OF THE INVENTION

The present invention relates to a complex utilizing one or more functional entity (FE), said FE(s) being capable of preventing degradation and/or removal of the complex, increasing the activity of the complex and/or increasing the transfer of the complex; extracellularly (within an organism), transcellularly (across a cellular membrane) and/or intracellularly (into different locations within a cell). The complex comprises a siRNA molecule, wherein the siRNA molecule comprises a first and a second strand and the siRNA molecule is modified in the following way:
at least one anchor-binding domain is incorporated into or attached to one of the two strands of the siRNA molecule, said anchor-binding domain being a nucleic acid or analog thereof,
at least one anchor sequence is hybridized to said anchor-binding domain, said anchor sequence being a nucleic acid or analog thereof, and
at least one functional entity (FE) is linked to said at least one anchor sequence, said FE(s) being one or more biologically active molecule.

In another embodiment of the present invention a short hairpin RNA is modified similarly to siRNA.

In yet another embodiment the present invention relates to an antisense (AS) molecule being modified in a similar fashion to siRNA/shRNA.

The present invention also relates to methods for producing the complex and for transferring the complex across a biological membrane in order to prevent degradation and/or removal of the complex, increase the activity of the complex and/or increase the transfer of the complex; extracellularly (within an organism), transcellularly (across a cellular membrane) and/or intracellularly (into different locations within a cell)

The present invention is defined in the following description and by the attached set of claims, hereby incorporated in its entirety.

### BRIEF DESCRIPTION OF THE DRAWINGS

FIG. 1 shows a schematic overview of the RNAi mechanism.

FIG. 2 is a schematic non-limiting illustration of a complex composed according to the present invention. Panel 2A shows a double stranded siRNA molecule. Panel 2B shows a double stranded siRNA molecule wherein an anchor-binding domain has been introduced or whereto an anchor-binding domain has been attached. Panel 2C shows a double stranded siRNA molecule wherein an anchor-binding domain has been introduced or whereto an anchor-binding domain has been attached and an anchor sequence to which a FE has been attached. Panel 2D shows a double stranded siRNA complex according to the present invention wherein the anchor sequence is attached to the anchor-binding domain. (Dotted lines indicate alternate embodiments.)

FIG. 3 shows a schematic non-limiting representation of an embodiment of the present invention wherein an anchor-binding domain is introduced into a shRNA molecule. The shRNA molecule is later cleaved into siRNA by Dicer. Hence, the hairpin loop and neighboring bases will be removed, indicated by arrowheads. (Dotted lines indicate alternate embodiments.)

FIG. 4 shows a schematic non-limiting representation of an AS (antisense) Bioplex according to the present invention. Panel 4A shows an AS molecule. Panel 4B shows an AS molecule to which an anchor-binding domain has been introduced. Panel 4C shows the attachment of an anchor-FE sequence to the AS molecule. Panel 4D shows the binding of the AS molecule to its target sequence. When the AS molecule hybridizes to its target sequence the anchor-FE sequence is released. (Dotted lines indicate alternate embodiments.)

FIG. 5 shows a schematic representation of an embodiment of the present invention wherein the complex comprises a cleavable linker. Left panel: A disulphide bridge is introduced within the anchor sequence. When reduced, the anchor is divided into two short anchor sequences with a too low ΔG to remain hybridized. Right panel: A disulphide bridge is introduced within the linker between the anchor and FE. When reduced, FE detaches and leaves siRNA-LNA triplex intact.

FIG. 6 shows an image of a PAGE shift assay of a competition assay between bis-PNA and LNA. The numbers represent the number of base pairs formed by RNA and either LNA or bisPNA respectively. Salt concentration indicated in mM.

FIG. 7 shows an image of a PAGE shift assay. Number of bases overlapping, is referring to the number of bases complementary to the target within the anchor-binding domain on the antisense strand.

FIG. 8 shows down regulation of Luciferase by siRNA. The plasmid holds the reporter gene luciferase. The bar named no siRNA represents transfection with the plasmid only, the other bars represent co-transfection of plasmid together with siRNA, as indicated. siRNA/Btk is an unrelated siRNA (i.e. lacking sequence similarity with the luciferase mRNA and should not affect the luciferase expression if the inhibition is specific).

FIG. 9 is a schematic representation of a complex according to the present invention used in the Experimentals. A functional entity (FE) is linked to a 7-mer LNA oligonucleotide anchor, hybridized to siRNA (e.g. S3). FIG. 10 shows an image of a PAGE retardation shift assay were the respective lanes are: 1: sense (S3s); 2: sense+0.8LNA (S3s+L5); 3: antisense (S3a); 4: siRNA (S3); 5: siRNA+0.8LNA (S3+L5); 6: siRNA+1.0LNA (S3+L5); 7: siRNA 1.2LNA (S3+L5); 8: siRNA+1.4LNA (S3+L5).

FIG. 11 shows down regulation of Luciferase. Average of triplicates and standard deviation.

FIG. 12 shows the results from the FACS analysis comparing the asialoglycoprotein receptor mediated uptake of siRNA (S3), S3 hybridized to LNA anchor only (L4) and S3 hybridized to L5, in HepG2 cells.

FIG. 13 shows siRNA uptake in the liver. A) S3 hybridized to L5, B) S3, C) No siRNA.

FIG. 14 shows an image of a PAGE retardation shift assay were the respective lanes are: 1: sense (S5s); 2: sense+0.8BPNA579 (S5s+P4); 3: antisense (S5a); 4: siRNA (S5); 5: siRNA+0.8PNA579 (S5+P4); 6: siRkNA+1.0PNA579 (S5+P4); 7: siRNA+1.2PNA579 (S5+P4); 8: siRNA+1.4PNA579 (S5+P4).

FIG. 15 shows a comparison of siRNAs sharing the same antisense sequence, targeting the Luciferase mRNA at the same position. Both the FE and the 3′ base substitutions of the sense strand influence the siRNA's down regulation ability. Normal siRNA (S1) is used as standard, values displays the increase or decrease of down regulation in comparison to S1. (Standard deviations indicated)

FIG. 16 is a schematic non-limiting illustration of an embodiment of the present invention wherein multiple FEs are attached to the same anchor through a branched linker (A); the anchor sequence is extended with an anchor-binding domain (B); and the anchor has a long extension with multiple anchor-binding domains (c).

FIG. 17. Panel A shows an image of a PAGE retardation shift assay were the respective lanes are: 1: S4; 2: S4+P6; 3: S4+P6+D7; 4: S4+P6+D7+L5; 5: S4/P6/D7/L5; 6: L5+D7+P6+S4; 7: L5+D7+P6; 8: L5+D7; 9: L5; 10: D7; 11: D7+P6. The constructs are added to the complex consecutively in the same order as written, Consecutive hybridizations (above) are separated by + and / indicates simultaneous addition. Panel B shows a schematic illustration of a complex as used example 11.

FIG. 18 shows the percentage of undegraded siRNA LucG2 (S3) only or hybridized with a LNA anchor having a trimeric carbohydrate as FE (S3+L5), at indicated time-points.

### DETAILED DESCRIPTION OF INVENTION

In the present description and claims, the following terms and abbreviations will be used:

The term “functional entity” (FE) relates to a biologically active molecule being any moiety capable of conferring one or more specific properties and/or biological functions to a molecule linked to it. As non-limiting examples, a FE linked to the complex according to the invention (or to any other molecule) can prevent degradation and/or removal (clearance) of the complex from its site of action, it can increase the activity of said complex, i. e. increase the down regulation of the gene, and/or increase the transfer of the complex to its site of action. The mode of action of a FE can be exerted extracellularly (within an organism), transcellularly (across a cellular membrane) and/or intracellularly (into different locations within a cell).

An “anchor” or “anchor sequence” may be any natural or synthetic nucleic acid, nucleic acid derivative or nucleic acid analog capable of specific, and optionally reversible, binding to a specified target thereof, preferably by hybridization. Non-limiting examples of anchors are PNA and LNA described below.

An “anchor-binding region” or “binding region” is a specific region corresponding to an anchor, and may be any natural or synthetic nucleic acid, nucleic acid derivative or nucleic acid analog capable of specific, and optionally reversible, binding to a specified anchor sequence, preferably by hybridization.

A “linker” (L) may be any chemical cleavable structure connecting, for example, a FE and an anchor. A cleavable linker can also be included in the anchor sequence or the sequence of the FE. Preferably the linker does not participate in the chemical/biochemical interactions of the FEs. The linker preferably resembles polyethylene glycol (PEG) or a natural or synthetic nucleic acid polymer, but may also be any suitable synthetic or natural-polymer.

“PNA” is an acronym for Peptide Nucleic Acid, which is a DNA mimic having a pseudopeptide backbone consisting of aminoethyl glycine units, to which the nucleobases are attached via methylen carbonyl linkers. A PNA molecule is capable of hybridizing to complementary ssDNA, dsDNA, RNA, PNA and other Watson-Crick and/or Hoogsteen base pairing oligonucleotide targets. The neutral backbone of PNA, in contrast to the negatively charged DNA, results in strong binding and without decrease in the high specificity. PNA was originally used for AS purposes but has found other fields of application. In the present application, it is to be understood that the term “PNA” refers to any DNA analog comprising the above backbone and nucleobases, and the term is thus not limited to the specific structures disclosed herein.

“LNA” is an acronym for Locked Nucleic Acid, which is also a DNA mimic. Locked Nucleic Acid (LNA) bases contain a bridging methylene carbon between the 2′ oxygen and 4′ carbon positions of the ribofuranose ring. This constraint preorganizes the oligonucleotide backbone and can increase Tₘ values by as much as 10° C. per LNA base replacement. LNA resembles natural nucleic acids with respect to Watson-Crick base pairing. LNA bases are introduced by standard DNA/RNA synthesis protocols, so LNA can be synthesized as pure LNA oligomers or mixed LNA/DNA/RNA oligomers. Moreover, LNAs have been demonstrated to be very efficient in binding to complementary nucleic acids and to be active antisense agents in vitro and in cultured mammalian cells, and also as decoys, aptamers, LNAzymes, and DNA correcting agents. In the present application, it is to be understood that the term “LNA” refers to any DNA analog comprising the above backbone and nucleobases, and the term is thus not limited to the specific structures disclosed herein.

The term “hybridize” refers to any binding or duplexing by base pairing of complementary bases of nucleic acids or any derivatives or analogs thereof.

The term “hairpin loop” relates to the region of duplex structure in RNA, formed by base pairing between adjacent or nearby complementary sequences on the same strand, the unpaired bases between the sequences forming a single stranded loop (hairpin loop). Hairpin loops can also be formed in DNA and other Watson-Crick base pairing oligonucleotides.

The present invention relates to a complex and methods of making such a complex in order to add functions in terms of delivery and specificity to RNAi through minimal modification of molecules possessing RNAi activity. The modified molecules have an unaffected or potentially increased RNAi activity.

Various functional entities are anchored through a DNA analog by Watson-Crick base pairing in a sequence specific manner to any one of the two strands of a double stranded RNA molecule. Preferably said RNA molecule is a siRNA or a shRNA molecule. In a similar approach, the present invention also presents a way to add functions in terms of delivery and specificity to antisense technology through minimal modification of molecules possessing antisense (AS) activity.

Further, the present invention relates to a platform for siRNA/shRNA delivery altered by the choice of functional entity. Functional entity(-ies) is/are added to the siRNA/shRNA molecule through DNA analog anchor(s). Chemical linkage of a functional entity directly to the siRNA would constrain the possibility to make a module setup combining different siRNAs and different functional entities, compatible in any combination. The different components of the inventive complex are synthesized separately and, for example, applying silencing of a reporter gene to an endogenous gene would only require a standard synthesis of siRNA. As a non-limiting example, functional entities can be tissue specific while the siRNAs are mRNA specific.

One embodiment of the present invention relates to a complex utilizing one or more functional entities (FEs), said FE(s) being capable of preventing degradation and/or removal of said complex, increasing the activity of said complex and/or increasing the transfer of said complex; extracellularly (within an organism), transcellularly (across a cellular membrane) and/or intracellularly (into different locations within a cell). Said complex comprises a siRNA molecule, wherein said siRNA molecule comprises a first and a second strand and said siRNA molecule is modified in the following way:
at least one anchor-binding domain is incorporated into or attached to one of the two strands of the siRNA molecule, said anchor-binding domain being a nucleic acid or analog thereof,
at least one anchor sequence is hybridized to said anchor-binding domain, said anchor sequence being a nucleic acid or analog thereof, and
at least one functional entity (FE) is linked to said at least one anchor sequence, said FE(s) being one or more biologically active molecule(s).

In the embodiments of the present invention relating to a complex based on a siRNA molecule, the term “increasing the activity of said complex” relates to the increase of the RNAi activity (i.e. the gene silencing effect).

The siRNA molecule should be double stranded RNA or analogs thereof, with a maximum length of about 35 base pairs, with or without overhangs. Preferably the siRNA is from 15 to 30 base pairs in length and more preferably from 17 to 25 base pairs in length. At least one of the 3′ and/or 5′-ends of the double stranded siRNA molecule is chosen as binding domain for the anchor. The length of the anchor-binding domain is determined by the sequence and the thermo dynamics of the anchor. A typical length of the anchor-binding domain is up to about 15 nucleotides, preferably about 3-12 nucleotides, and more preferably about 4-8 nucleotides.

The nucleotides of the anchor-binding domain can be within the original siRNA molecule or as an extension at any one of the two ends of the chosen strand. In one embodiment of the present invention the anchor-binding domain is incorporated in the sense strand of the siRNA molecule and in another embodiment in the antisense strand.

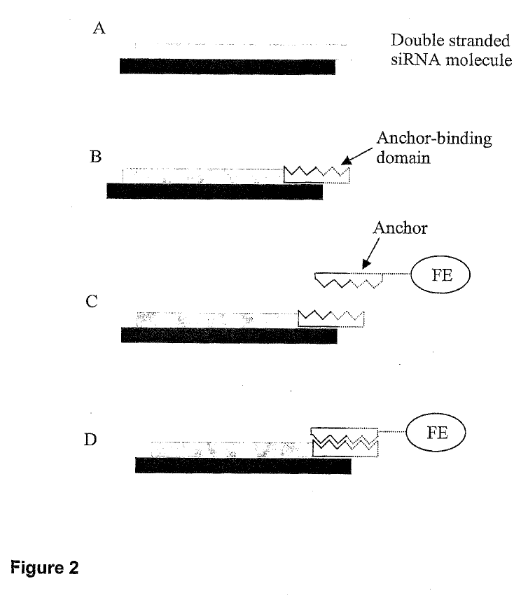

FIG. 2 shows one embodiment of a complex according to the present invention and how such a complex is composed and the binding of an anchor to the anchor-binding domain introduced into a siRNA molecule.

Choosing the 3′-end of the sense strand as anchor-binding domain might be advantageous, since the sense strand in a normal siRNA molecule is suggested to be for recruiting RISC. The RISC will then dissociate the strands and route the antisense strand to the correct target RNA or DNA. Studies have shown that RISC preferentially attacks the weakest end of the siRNA in terms of ΔG and binds to whichever strand that has its 5′-end open. When an anchor is hybridized to the 3′-end of the sense strand, RISC would bind to the 5′-end of the antisense strand.

To further increase the hybridization efficacy of the anchor, the nucleotides of the anchor-binding domain may have one or more mismatches with respect to the second strand. By introducing mismatches, competition can be reduced between the anchor and the strand of the RNA molecule not possessing the anchor-binding domain. Preferably, the nucleotides of the anchor-binding domain may have 1-7 mismatches with respect to the other strand and preferably 2-5 mismatches.

In one embodiment of the present invention the hybridization of the anchor to the anchor-binding domain is reversible. The Watson-Crick base pairing between the anchor-binding domain and the anchor relies on hydrogen bonds between the bases. If desirable, it is possible to make the anchor fall off during the intracellular processing by decreasing the length of the anchor. The binding affinity is high enough at physiological conditions for stable hybrids of siRNA-anchor-FE to form when they do not compete for binding with other molecules. However, proteins and other molecules with affinity for siRNA will compete with these weak bonds leading to release of the anchor-FE complex. Covalent linkage of a biologically active molecule directly to the RNA molecule would lack this instability and possibly hinder interactions needed for gene silencing.

In a non-limiting example the anchor-binding domain is 7 bases, the 3′-end of the sense strand has been extended with one base of choice. Thus, the anchor-binding domain constitutes of 4 bases within the duplex and 3 bases overhang. To further increase the hybridization efficacy between anchor-binding domain and anchor, mismatches are preferentially introduced to reduce or abolish the competition between the antisense strand and the anchor.

The anchor can be any nucleic acid or analog thereof capable of hybridization to the anchor-binding domain. In one embodiment the anchor has a bridge between 2′ and 4′ within the ribofuranose ring to stabilize C3′-endo/N-type sugar conformation. Such anchors possess enhanced thermo stability when forming base pairs with RNA according to the Watson-Crick or Hoogsteen model. Since short anchors are preferred (maximum 15 bases), Tm is an important property of the anchor of choice. Examples of suitable anchors are, but not limited to, locked nucleic acid (LNA), 2′-O, 4′-C-ethylene bridged nucleic acid (ENA), bridge nucleic acid (BNA) or analogs thereof. Other candidates with a completely different backbone are deoxyribonucleic guanidine (DNG), morpholino oligos and peptide nucleic acid (PNA), also exhibiting sequence specific base pairing. Intercalators could also be used to increase binding affinity, preferable in combination with DNA analogs. Examples of intercalators are Intercalating nucleic acid (INA) and acridine.

In one particular embodiment of the present invention LNA is used as anchor sequence. At physiological conditions, only 7 bases are required for stable hybrid formation with single stranded RNA (ssRNA). The sense strand is mismatched with respect to the antisense strand within the binding region, positioned at the 3′ end of the sense strand. The binding domain comprises a 3 bases overhang and 4 bases into the double strand.

In one embodiment of the present invention the siRNA molecule has 3′ and/or 5′ overhangs of about 1-12 nucleotides, preferably about 2 to 5 nucleotides.

In yet another embodiment of the present invention the siRNA molecule is chemically modified. Examples of chemical modifications of the sense and/or the antisense strand are, but not limited to, one or more 2′-O-methyl modified pyrimidine nucleotides, one or more 2′-deoxy-2′-fluoro modified pyrimidine nucleotides and at least one phosphorothioate internucleotide linkage at the5′ or 3′ end. Further chemical modifications of the siRNA molecule including any overhangs are routine and obvious for a person skilled in the art.

At least one functional entity (FE) is linked to the anchor. As described in the Bioplex patent (WO 00/15824, which is hereby incorporated by reference in its entirety, including any drawings), FE could be any amino acid peptide, carbohydrate, lipid, nucleic acid or other molecule with biological activity and combinations thereof, providing any number of functions such as, but not limited to, a structural function e.g. binding to a cell membrane target molecule or an enzymatic function. More specific non limiting examples are the function of cellular attachment (e.g. electrostatic attraction with polycationic FE), cell internalization (e.g. transferrin, the tripeptide RGD, TAT, transportan and other cell penetrating peptides (CPGs)), endosomal escape (fusion proteins e.g. HA2) and in the case of post-translational gene silencing, nuclear transport (e.g. nuclear localization signal). More specifically, for liver uptake, a tri-antennary N-acetylated galactose amine can be used for uptake into hepatocytes through the asialoglycoprotein receptor.

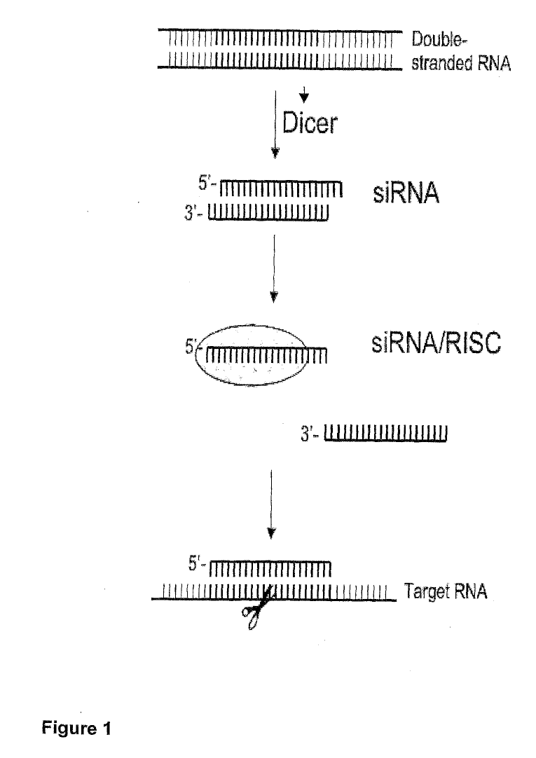

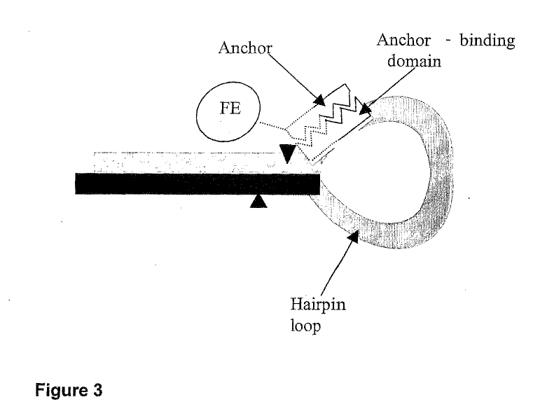

In another embodiment of the present invention a shRNA molecule can be used in the inventive complex (see FIG. 3). A shRNA is a short sequence of RNA which makes a tight hairpin loop and can be used to silence gene expression. shRNA are usually shorter than 80 nucleotides, preferably not more than 50 nucleotides in length, and more preferably not more than 35 nucleotides in length, with the hairpin loop constituting about 2-12 of these nucleotides. Dicer will recognize shRNA in a fashion resembling double stranded RNA and cleave it at predictable positions, generating short double stranded RNA of typically 19-22 base pairs with 2 bases overhang at the 3′ ends. RISC will then disrupt the duplex and bind one of the strands. The mechanism is depicted in FIG. 1, with the exception of the presence of a hairpin loop. It has been suggested that Dicer activity will potentiate the recruitment of RISC to siRNA and thereby increase the gene silencing efficacy.

By modifying the shRNA sequence, specificity and functionality can be added to shRNA in the same manner as to the siRNA molecule. The same technique that is applied for siRNA can be used for modification of the hairpin loop to act as an anchor-binding domain. Since Dicer will cleave the shRNA at predictable positions, the hairpin (possibly along with additional bases depending on design) will be cleaved off and not be a part of the gene silencing process. Thus, the sequence that is to be part of the gene silencing process can be kept fully matched or mutated, mismatched or designed in other ways.

Thus, one embodiment of the present invention relates to a complex utilizing one or more functional entities (FEs), said FE(s) being capable of preventing degradation and/or removal of said complex, increasing the activity of said complex and/or increasing the transfer of said complex; extracellularly (within an organism), transcellularly (across a cellular membrane) and/or intracellularly (into different locations within a cell). The complex comprises a short hairpin RNA (shRNA) molecule, wherein said shRNA molecule has a double stranded region and a hairpin loop and said shRNA molecule is modified in the following way:
at least one anchor-binding domain is incorporated into the shRNA molecule, said anchor-binding domain being a nucleic acid or analog thereof,
at least one anchor sequence is hybridized to said anchor-binding domain, said anchor sequence being a nucleic acid or analog thereof, and
at least one functional entity (FE) is linked to said at least one anchor sequence, said FE(s) being one or more biologically active molecule(s).

In the embodiments of the present invention relating to a complex based on a shRNA molecule, the term “increasing the activity of said complex” relates to the increase of the RNAi activity.

The length of the anchor-binding domain is determined by the sequence and the thermo dynamics of the anchor. A typical length of the anchor-binding domain is up to about 15 nucleotides, preferably about 3-12 nucleotides, and more preferably about 4-8 nucleotides.

Hairpin loops of the shRNA can be used as anchor-binding domains without any modifications or the hairpin can be changed to any suitable sequence with respect to anchor sequences available. Only a part or the entire loop can be used as an anchor-binding domain but the anchor-binding domain could also be partly in the double stranded region and partly in the hairpin loop or entirely incorporated in the double stranded region. The anchor-binding domain could furthermore be positioned at the 3′ and/or5′ end of the shRNA or anywhere in the double stranded region of the shRNA. Thus, the anchor binding domain can be a part of the shRNA molecule but also an extension at either end of the shRNA molecule. Preferably, when having the anchor-binding domain within the double stranded region of the shRNA, mismatches should be introduced as described for siRNA.

In one embodiment of the present invention the shRNA molecule has 3′ and/or 5′ overhangs of about 1-12 nucleotides, preferably about 2 to 5 nucleotides. The shRNA complex can also be chemically modified as described for the siRNA molecule.

Further characteristics of the anchor, anchor-binding domain and FE described for the inventive complex based on siRNA also apply for the inventive complex based on shRNA.

FIG. 3 shows a schematic illustration of a non-limiting example of a modification of shRNA according to the present invention. The shRNA will be cleaved into siRNA by Dicer. Hence, the hairpin loop and neighboring bases will be removed as indicated by arrowheads. The region that is to be cleaved off can therefore be modified to act as binding domain.

In addition to RNAi, this invention can also be applied on antisense technology. To reduce the risk of confusion, antisense as a technique is referred to as “AS”, the antisense strand as a component of siRNA/shRNA will still be denoted “antisense”.

AS is a single stranded DNA oligonucleotide (or analog thereof), which is designed to bind to the RNA of the gene to be silenced. The cause of action is in this case not digestion of the RNA but simply hybrid formation and hindrance of further processing of the RNA. In a specific situation an AS is composed of single stranded RNA instead of DNA with a RISC independent degradation of the target RNA by the recognition of dsRNA by RNases as result. AS might also be capable of DNA and histone methylation in a similar way as siRNA, hence the target could possibly also be DNA.

Thus, in another embodiment the present invention relates to a complex utilizing one or more functional entities (FEs), said FE(s) being capable of preventing degradation and/or removal of said complex, increasing the activity of said complex and/or increasing the transfer of said complex; extracellularly (within an organism), transcellularly (across a cellular membrane) and/or intracellularly (into different locations within a cell). Said complex comprises an antisense (AS) molecule, wherein said AS molecule is modified in the following way:
at least one anchor-binding domain is incorporated or attached to the AS molecule, said anchor-binding domain being a nucleic acid or analog thereof,
at least one anchor sequence is hybridized to said anchor-binding domain, said anchor sequence being a nucleic acid or analog thereof, and
at least one functional entity (FE) is linked to said at least one anchor sequence, said FE(s) being one or more biologically active molecule(s).

In the embodiments of the present invention relating to a complex based on an AS molecule, the term “increasing the activity of said complex” relates to the increase of the gene silencing effect.

The length of the AS molecule is usually about 15-35 nucleotides and preferably about 17-25 nucleotides. The anchor sequence could either be chosen to bind to the AS or to an extension of the AS sequence, i.e. the anchor-binding domain can be a part of the AS molecule, at any position, or an extension at either end of AS molecule.

The length of the anchor-binding domain of the AS molecule is determined by the sequence and the thermodynamics of the anchor. A typical length of the anchor-binding domain is up to about 15 nucleotides, preferably about 3-12 nucleotides, and more preferably about 4-8 nucleotides. Having a short anchor-binding domain and anchor sequence will give a specific release of the FE when the AS hybridizes to its target (e.g. RNA). The DNA-RNA hybrid has a melting temperature exceeding that of the anchor-AS due to a higher number of hybridizing base pairs in the DNA-RNA hybrid. The short anchor will be too weak to interfere with, for example, chromosomal DNA. No mismatches are introduced in the anchor-binding domain with respect to the anchor sequence or to the target region. However, if the anchor-binding domain is an extension of the AS, the anchor-binding domain could be mismatched with respect to the RNA. Mismatches would probably not result in controlled release of the anchor upon hybridization to the target RNA.

The characteristics of the anchor sequence hybridizing to the AS molecule and the FE(s) attached to the anchor sequence are the same as for the embodiments comprising siRNA and shRNA.

Similar to the siRNA and shRNA molecule, the AS molecule can be chemically modified. Chemical modification of the AS molecule is routine and obvious for a person skilled in the art.

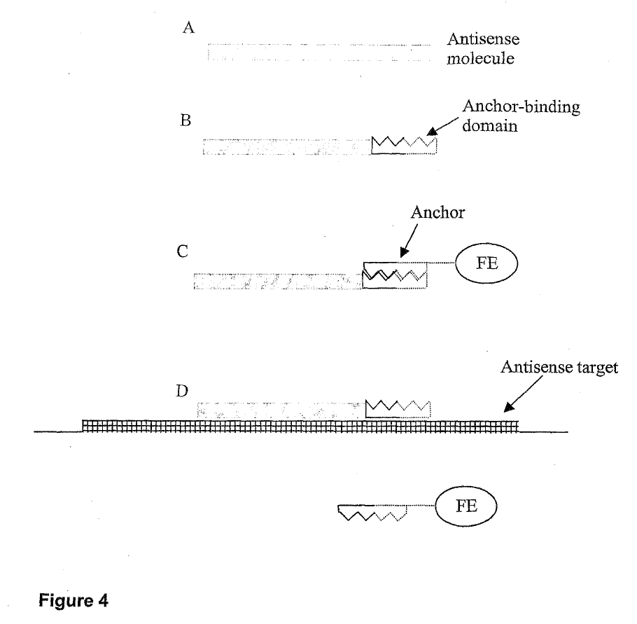

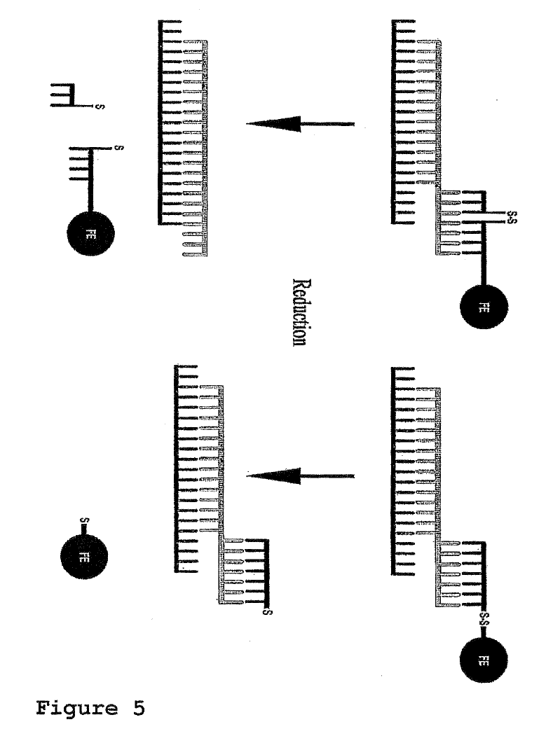

FIG. 4 is a schematic non-limiting illustration of an AS Bioplex with release of the FE upon binding of the AS molecule to the target.

In yet another embodiment of the present invention the anchor and/or FE can be released from the inventive complex (based on siRNA, shRNA or AS) through the introduction of cleavable linkers. Thus, if RISC is inhibited by the FE or the anchor, this problem could be solved by cleaving the linker(s) inserted in the complex. There are different variants of cleavable linkers (se also the disclosure of WO 03/091443, which is hereby incorporated by reference in its entirety, including any drawings).

The cleavable linker molecule(s) can be introduced between the FE(s) and the anchor sequence(s) or, optionally, introduced within the anchor sequence(s).

One non-limiting example of a linker that can be used is a disulphide bridge. Since the siRNA/shRNA molecule probably would end up in the cytoplasm before RISC assembly, the reducing milieu in the cytoplasm will reduce the sulphide and break the bridge.

FIG. 5 is a schematic non-limiting example of the release of an anchor and/or a FE through reduction of a cleavable linker. The left panel shows a disulphide bridge introduced within the anchor sequence. When reduced, e.g. through cytoplasmic reduction, the anchor is divided into two short anchor sequences with a too low ΔG to remain hybridized. The right panel shows a disulphide bridge introduced within the linker between the anchor and the FE.

Increasing thermal stability between the two strands in siRNA and within the self-complementary region of shRNA could be used for RISC guidance and/or to increase the affinity for the target RNA. Also AS potency can be increased by increased binding affinity to the target RNA. Increased affinity towards target RNA and internal stability of siRNA and shRNA can be obtained through nucleotide substitutions of RNA or DNA with DNA analougs (PNA, ENA, BNA, LNA etc.) or by intercalators (INA, Acridin etc.)

The anchor-binding domain can also have DNA nucleotides substituted with analogs with stronger hybridizing properties for increased stability of the binding of the anchor and/or to for maintaining the anchor sequence as short as possible. There are reports of LNA-LNA duplexes of as short as 4 base pairs at physiological conditions.

Bioplex was first developed for plasmids where the number of bases is not limiting (see patent applications Nos. WO 00/15824 and WO03/091443, which are hereby incorporated by reference). The maximum length of a siRNA is preferably a 35 base pair duplex with a few bases overhang at each ends. Others have shown the Tm of siRNA to be of major importance for gene silencing.

Thus, there are only a few bases that can be used to incorporate an anchor-binding binding domain, a stable siRNA duplex silencing domain that should be accessible for RISC (and/or other proteins). It might be of interest to have several different FEs coupled to the same siRNA in order to give the siRNA different functions within the cell or the biological organism. Similarly, it might be of interest to have several different FEs coupled to the same shRNA or AS molecule in order to give the shRNA/AS different functions within the cell or the biological organism.

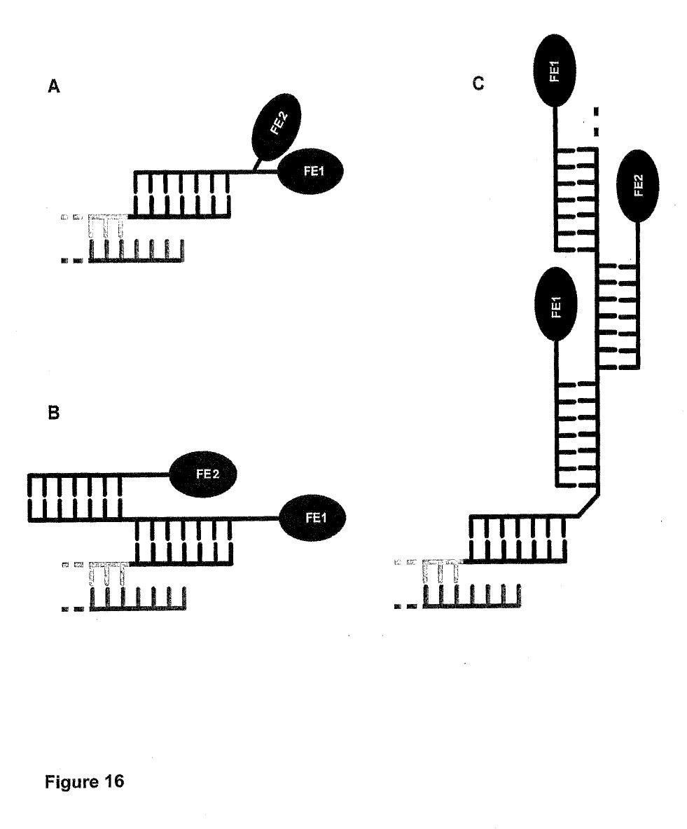

There are multiple ways of adding multiple FEs to one anchor sequence. First the FEs can be branched from the anchor (FIG. 16A). This would give a well characterized and defined complex. The platform would however be less flexible. Another approach is to extend the anchor sequence. This would provide an anchor-binding domain for the next anchor and its FE (FIG. 16B), yielding a module system were FEs easily can be combined, making the platform more flexible.

FIG. 16C illustrates a further refinement of this module system giving multiple FE combinations, all with FE specific anchor sequences. The anchor would not have a FE but rather be a long oligonucleotide, linear or branched, with an anchor region and multiple anchor-binding domains. The oligonucleotide can contain modified or unmodified bases (DNA analogs) or a mixture of both.

The siRNA, shRNA and AS molecule can be modified similarly to the illustrative molecule depicted in FIG. 16.

The present invention also relates to a method for making a complex that utilises one or more functional entities (FEs), said FE(s) being capable of preventing degradation and/or removal of said complex, increasing the activity of said complex and/or increasing the transfer of said complex; extracellularly (within an organism), transcellularly (across a cellular membrane) and/or intracellularly (into different locations within a cell). The complex comprises a short interfering RNA (siRNA) molecule and said siRNA molecule comprises a first and a second strand. The method comprises the following steps:
introducing at least one anchor-binding domain into the siRNA molecule, said anchor-binding domain being a nucleic acid or analog thereof,
hybridizing at least one anchor sequence to said anchor-binding domain, said anchor sequence being a nucleic acid or analog thereof, and
linking at least one functional entity (FE) to said at least one anchor sequence, said FE(s) being one or more biologically active molecule(s).

The anchor-binding domain can be introduced into any one of the two strands of the siRNA molecule.

Further, the present invention also relates to a method for making a complex that utilises one or more functional entities (FEs), said FE(s) being capable of preventing degradation and/or removal of said complex, increasing the activity of said complex and/or increasing the transfer of said complex; extracellularly (within an organism), transcellularly (across a cellular membrane) and/or intracellularly (into different locations within a cell). The complex comprises a short hairpin RNA (shRNA) molecule and the shRNA molecule has a double stranded region and a hairpin loop. The method comprises the following steps:
introducing at least one anchor-binding domain into the shRNA molecule, said anchor-binding domain being a nucleic acid or analog thereof,
hybridizing at least one anchor sequence to said anchor-binding domain, said anchor sequence being a nucleic acid or analog thereof, and
linking at least one functional entity (FE) to said at least one anchor sequence, said FE(s) being one or more biologically active molecule(s).

The characteristics of the complex based on a siRNA and shRNA molecule, respectively, described above also apply for the complex in the methods of making such a complex.

In another embodiment the present invention relates to a method for making a complex that utilises one or more functional entities (FEs), said FE(s) being capable of preventing degradation and/or removal of said complex, increasing the activity of said complex and/or increasing the transfer of said complex; extracellularly (within an organism), transcellularly (across a cellular membrane) and/or intracellularly (into different locations within a cell). The complex comprises an AS molecule. The method comprises the following steps;
incorporating or attaching at least one anchor-binding domain to the AS molecule, said anchor-binding domain being a nucleic acid or analog thereof,
hybridizing at least one anchor sequence is to said anchor-binding domain, said anchor sequence being a nucleic acid or analog thereof, and
linking at least one functional entity (FE) to said at least one anchor sequence, said FE(s) being one or more biologically active molecule(s).

The characteristics of the complex based on an AS molecule described above also apply for the complex in the method of making such a complex.

The present invention also relates to a method for transferring one or more FEs across a cellular membrane and into different locations within a cell wherein a complex based on an AS molecule as described above is used for transfection. In particular, the method comprises the following steps;
incorporating or attaching at least one anchor-binding domain to an AS molecule, said anchor-binding domain being a nucleic acid or analog thereof,
hybridizing at least one anchor sequence is to said anchor-binding domain, said anchor sequence being a nucleic acid or analog thereof,
linking at least one functional entity (FE) to said at least one anchor sequence, said FE(s) being one or more biologically active molecule(s), and
contacting said complex with a cell or biological membrane.

The characteristics of the complex based on a AS molecule described above also apply for the complex in the method of making such a complex and in the method of transferring said complex across a cellular membrane in order to prevent degradation and/or removal of said complex, increase the activity of said complex and/or increase the transfer of said complex; extracellularly (within an organism), transcellularly (across a cellular membrane) and/or intracellularly (into different locations within a cell).

The present invention also relates to a method for transferring one or more FEs across a cellular membrane and into different locations within a cell wherein a complex based on a RNA molecule as described above is used for transfection in order to prevent degradation and/or removal of said complex, increase the activity of said complex and/or increase the transfer of said complex; extracellularly (within an organism), transcellularly (across a cellular membrane) and/or intracellularly (into different locations within a cell). The method comprises the following steps:
introducing at least one anchor-binding domain into a RNA molecule, said anchor-binding domain being a nucleic acid or analog thereof,
hybridizing at least one anchor sequence to said anchor-binding domain, said anchor sequence being a nucleic acid or analog thereof, and
linking at least one functional entity (FE) to said at least one anchor sequence, said FE(s) being one or more biologically active molecule(s),
contacting said complex with a cell or biological membrane; and
   wherein said RNA molecule is a siRNA or a shRNA molecule.

The characteristics of the complex based on a siRNA and a shRNA molecule, respectively, described above also apply for the complex in the method of transferring said complex across a cellular membrane and into different locations within a cell.

The present invention also relates to a cell transfected in vitro or in vivo with a complex as described above. The cell can be a eukaryotic or prokaryotic cell. The eukaryotic cell may be a human or non-human cell.

The complex according to the present invention can be introduced into cells in a number of ways, such as, but not limited to, micro-injection, soaking the cell or organism in a solution comprising the inventive complex, electroporation of cell membranes in the presence of the inventive complex, liposome mediated delivery of the inventive complex, transfection and transformation. The inventive complex can also be introduced together with other components enhancing uptake of said complex.

Furthermore, the invention also relates to a kit comprising components for producing a complex as described above. More specifically, the present invention relates to a kit comprising components for producing a complex capable of transferring one or more siRNA, shRNA or AS molecules across a cellular membrane (intracellularly, extracellularly and/or transcellularly) and into different locations within a cell, said kit comprising at least one siRNA, shRNA and/or AS molecule, at least one anchor-binding domain, at least one anchor sequence being able to hybridize to said anchor-binding domain, and at least one functional entity (FE) linked to said anchor-binding domain, said FE being one or more biologically active molecule(s).

In summary, the present invention presents a complex and a method of making such a complex in order to add functions in terms of delivery and specificity to RNAi with minimal or no effect on the binding of the RISC complex. The RNA molecules (siRNA or shRNA) can be modified without chemical modification and thus synthesized as regular siRNA/shRNA molecules. Surprisingly, there is no need of long extensions of the RNA molecules in order for the anchor sequence to attach, but introduction of a few mismatches is enough. Unmodified siRNA is rather unspecific at the cellular level, thus through attachment of biologically active molecule(s) the present invention will confer cell specificity to RNAi and possibly also down regulation of other proteins. Introduction of cell specificity also introduces a security aspect to RNAi.

### Experimentals

Materials and Methods Employed in Examples 1-10.

TABLE 1
Constructs and oligonucleotides. Lower case indicates amino acids.
L is a short PEG like linker (AEEA).

| | SEQ | |
|---|---|---|
| LNA constructs | name | SEQ ID. NO. |
| LNA1: 5′-CCCCT | L1 | SEQ ID No. 1 |
| LNA2: 5′-CCCCTT | L2 | SEQ ID No. 2 |
| LNA3: 5′-CCCCTTT | L3 | SEQ ID No. 3 |
| LNA-G2: 5′-ACC GAC C | L4 | SEQ ID No. 4 |
| 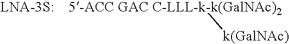 | L5 | 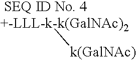 |
| siRNAs | | |
| siRNA LucNorm | S1 | |
| sense: CUU ACG CUG AGU ACU UCG AdTdT | S1s | SEQ ID No. 5 |
| antisense: UCG AAG UAC UCA GCG UAA GdTdT | S1a | SEQ ID No. 6 |
| siRNA LucExt | S2 | |
| sense: CUU ACG CUG AGU ACU UCG A AAAGGGG | S2s | SEQ ID No. 7 |
| antisense: UCG AAG UAC UCA GCG UAA GTT | S2a | SEQ ID No. 8 |
| siRNA LucG2 | S3 | |
| sense: CUU ACG CUG AGU ACU AA GGUCGGdT | S3s | SEQ ID No. 9 |
| antisense: UCG AAG UAC UCA GCG UAA GTT | S3a | SEQ ID No. 10 |
| siRNA siLucGsite | S4 | |
| sense: CUU ACG CUG AGU ACU AAA AGA AGA A | S4s | SEQ ID No. 11 |
| antisense: UCG AAG UAC UCA GCG UAA GTT | S4a | SEQ ID No. 12 |
| DNA oligonucleotides | | |
| antisense LucG2: 5′-CTT ACG CTG AGT ACT AA | D1 | SEQ ID No. 13 |
| GGTCGGdT | | |
| Target1over: 5′-C TT AGT ACT CTA CGT AAG | D2 | SEQ ID No. 14 |
| Target2over: 5′-CC TT AGT ACT CTA CGT AAG | D3 | SEQ ID No. 15 |
| Target3over: 5′-ACC TT AGT ACT CTA CGT AAG | D4 | SEQ ID No. 16 |
| DNA-decoy: 5′-AGA TCA GAC CGG ATC | D5 | SEQ ID No. 17 |
| siDNA siLucGsite | D6 | |
| sense: CUU ACG CUG AGU ACU AAA AGA AGA A | D6s | SEQ ID No. 18 |
| antisense: UCG AAG UAC UCA GCG UAA GTT | D6a | SEQ ID No. 19 |
| Carrier-2S: | D7 | SEQ ID No. 24 |
| TGT ACG TCA CAA CTA TTG GTC GGT TTG GTC GGT | | |
| PNA constructs | | |
| MCG1: N′- CCCCT-LLL-TCCCC-C′ | P1 | SEQ ID No.1-LLL- SEQ ID No.1 |
| MCG2: N′- CCCCTT-LLL-TTCCCC-C′ | P2 | SEQ ID No.2-LLL- SEQ ID No.2 |
| MCG3: N′- CCCCTTT-LLL-TTTCCCC-C′ | P3 | SEQ ID No.3-LLL- SEQ ID No.3 |
| PNA579: kkkLLTTCTTCTTTTLLLTTTTCTTCTTLLpkkkrkv | P4 | kkkLL-SEQ ID No.20-LLL- |
| | | SEQ ID No.20-LL- SEQ ID |
| | | No.21 |
| PNA580: kkkLLTTCTTCTTTTLLLTTTTCTTCTTLLvkrkkkp | P5 | kkkLL-SEQ ID No.20-LLL- |
| | | SEQ ID No.20-LL- SEQ ID |
| | | No.22 |
| PNA2946: | P6 | k-SEQ ID No.20-kkkLLLL- |
| kTTCTTCTTTTLLLTTTTCTTCTTkkkLLLLACATGCAGTGTTGAT | | SEQ ID No. 23 |

DNA oligonucleotides were purchased from DNA Technology A/S, Aarhus, Denmark. PNAs were purchased from Eurogentec S. A, Seraing, Belgium. siRNAs purchased from CureVac, Germany. Synthesis of GalNAc and its conjugation to LNA oligomer is described in detail by Westerlind et al., Glycoconj J. 2004;21(5):227-41.

The Reason for Using DNA Oligonucleotides

For many of the test tube studies, it is convenient to work with DNA oligonucleotides instead of RNA. DNA is cheaper, more stable and is synthesized and shipped within a few days.

When DNA is used instead of RNA, LNA hybrid formation is disfavoured. LNA has higher binding affinity towards RNA than DNA, the difference is in the order of 2-5° C. PNA, on the other hand, gains a few degrees in binding affinity as compared to RNA. I.e. hybridization to DNA will be even more stable with RNA. In example 1, LNA is chosen as anchor for further development of the Bioplex siRNA platform.

In terms of uptake, both RNA and DNA have a negatively charged backbone and will be repelled by the cell membrane. We therefore suggest that DNA and RNA are affecting the receptor-mediated endocytosis of presented FE in a similar fashion.

The term “siDNA” refers to a double stranded DNA oligonucleotide that has the same design as the siRNA it is designed to mimic, the difference being DNA instead of RNA nucleotides (i.e. thymidine instead of uracil).

Complex Assembly

Synthetic siRNA was annealed according to manufacturers protocol together with supplied annealing buffer (CureVac, Tübingen, Germany).

Anchors, of LNA or PNA with or without FEs, are added in excess with respect to the number of anchor-binding domains. 1.2 times excess of anchors as compared to the number of anchor-binding domain has been used to guarantee total occupation of the anchor-binding domains. The siRNAs and the DNA oligonucleotides used have only had one anchor-binding domain, i.e. 1 mol siRNA is hybridized with 1.2 mol of anchors. siRNAs or DNA oligonucleotides are incubated for half an hour at 37° C. under physiological conditions (Rignes buffer, 147 mM NaCl, 4 mM KCl₂ and 1.13 mM CaCl₂, pH 7.2). Final concentration of LNA/PNA is typically between 1-10 μM.

PAGE Retardation Shift Assay

Hybrid formation was verified on 20-% non-denaturing PAGE (polyacrylamide gel electrophoresis, TBE buffer [89 mM Tris, 89 mM Boric acid, 2 mM EDTA, pH8.3]) for 3 h at 90V. Fluorescently labeled oligonucleotides were detected without any staining, unlabeled nucleotides were stained with Sybr Green (Invitrogen) according to the protocol supplied by the manufacturer. Gels were scanned with a BioRad Molecular Imager FX pro plus (BioRad, Hercules, USA), with laser and filter settings for discrimination between the different dyes.

Luciferase Assay

Luciferase assay kit was purchased form BioThema (Haninge, Sweden), including protocol. The cells were rinsed with PBS (phosphor buffered saline, Invitrogen) and lysed with Reporter lysis buffer (Promega Corpration, WI, USA), 20 μL Reporter Lysis was diluted with 80 μL water and added to the cells which were then freezed and thawed once. 10 μL of lysed sample was added to a microplate well. Before loading the plate into the emission reader, 100 μL of reconstituted Luciferin Substrate was added to each well. The same volume of reconstituted ATP Substrate was added automatically by the apparatus. The light emission was measured with FLUOstar Optima (BMG Labtech, Offenburg, Germany).

### EXAMPLE 1

### Comparing DNA and PNA Analog Suitability as Anchor.

The bis-PNAs (Seq: P1, P2 and P3) hybridization efficacy to DNA oligonucleotides was compared to LNA (Seq: L1, L2 and L3). Hybridization was performed in varying salt concentrations, ranging from 0-150 mM NaCl. After one hour incubation at 37° C. with LNA and/or bisPNA and siRNA LucExt (S2), the samples were run on PAGE (described earlier). Hybrids with LNA will migrate slower than siRNA without LNA and bisPNA-siRNA hybrids will migrate even slower. Thus, a PAGE retardation shift assay shows which one of the analogs that forms a hybrid with the siRNA.

When a LNA and bis-PNA compete for hybridization to the anchor-binding domain of the extended siRNA (S2), a 6mer LNA (L2) oligonucleotide show higher binding affinity then a 7mer bis-PNA (P3) at physiological salt concentration.

For the specific siRNA sequence (LucExt (S2)) used and its corresponding anchor sequence, an anchor length of 5 bases (L1) were found to be the limit for hybridization (i.e. 5 bases were not enough), whereas 6 bases (L2) were sufficient for hybrid formation between the single stranded part of the siRNA and LNA at physiologic salt concentration. With a 6mer anchor sequence there might be competing bases (sense and antisense forming Watson-crick base pairing instead of sense and LNA) and at a later stage a functional moiety should be conjugated to the LNA. Taken this in consideration a 7mer LNA sequence was chosen as an anchor in further experiments.

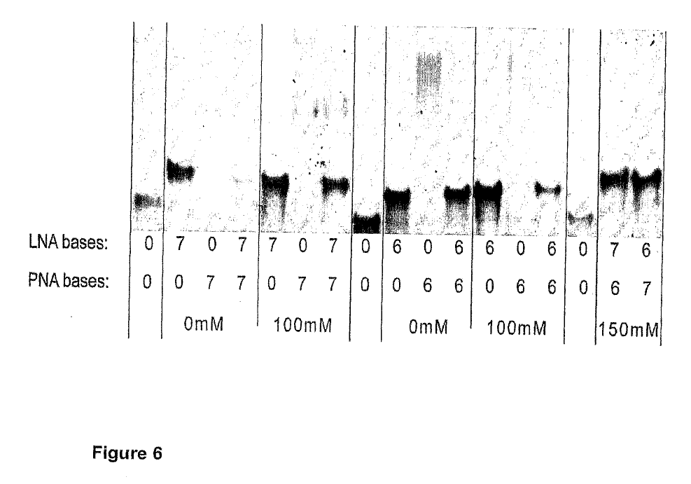

FIG. 6 shows an image of a PAGE of a competition assay for hybrid formation with RNA. DNA was also used as target with similar outcome. We choose to continue with developing the bioplex siRNA with LNA as anchor: This gives a total freedom in choice of sequence and a very short anchor-binding domain is needed. Bis-PNA is restricted to homo-pyrimidines only since it hybridizes through both Watson-Crick and Hoogsteen base pairing. In addition, this dual type of base pairing requires longer synthesis and is therefore more expensive.

Various design strategies utilizing two different anchors, LNA and bis-PNA, had now been tried. LNA seemed to be a better candidate under our experimental conditions because of the very short anchor-binding domain needed and the total freedom in choice of sequence.

### EXAMPLE 2

### Controlled Release of Functional Entity from AS.

A LNA anchor with 7 bases was chosen. Since LNA is not restricted in the composition as bisPNA is, the sequence could be chosen freely. In order to fulfill the siRNA design recommendations, an attempt was made to move the binding site “into” the double stranded part of the siRNA. However, it was not known whether LNA would sustain the competition by the antisense strand. A set of siDNAs were synthesized where the antisense strand was mismatched at one or multiple positions with respect to the sense strand. Thus, the LNA would have to compete against fewer bases.

If the sense and antisense strands are fully matching, our 7mer LNA (ACCGTCCA, L4) would not bind. We elucidated how many mismatches are needed for a stable complex and found that 2 out of 4 could be accepted depending on where they are positioned. However, if the LNA hybridizes to the anchor-binding domain, no base pairing between the sense and the antisense strand of the siRNA will be possible in the anchor-binding domain.

Low tolerance for competing bases gives the potential of having a controlled release of the anchor sequence together with the FE from the AS. When the single stranded DNA antisense molecule finds it target mRNA, the anchor is anticipated to fall off due to the weak hybridization of only seven bases.

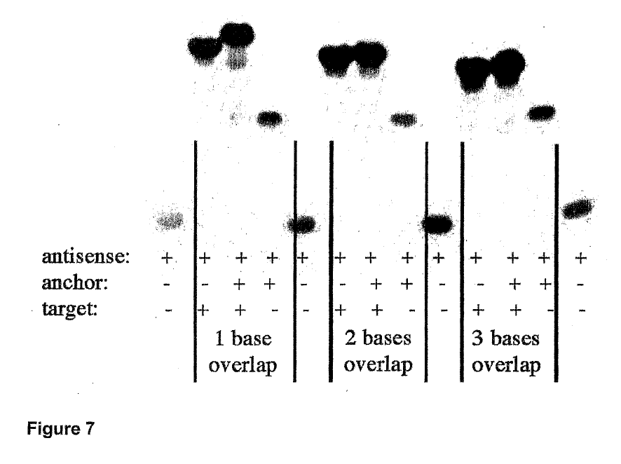

Antisense (D1) and anchor (L4) was hybridized as previously described. The target (D2, D3 or D4) was added at an equimolar ratio and incubated in Rignes salt solution at 37° C. for an additional hour. The PAGE retardation assay (described earlier) shows that if more than one of the bases in the anchor-binding domain of the antisense is complementary to the target, the anchor will be released from the antisense in benefit for the target (see FIG. 7).

### EXAMPLE 3

### Gene Silencing Efficiency of Modified siRNA

100 000 HepG2 cells were seeded in each well of a 24 well plate. After overnight incubation, the cells were transfected with 0.3 μg of reporter plasmid using Lipofectamine, according to the protocol for plasmid transfection supplied by the manufacturer, (Invitrogen) and incubated at 37° C. over night. The cells were then transfected with 20 pmol siRNA using Lipofectamine, according to the protocol for siRNA transfection supplied by the manufacturer, and down regulation was measured 72 hours later.

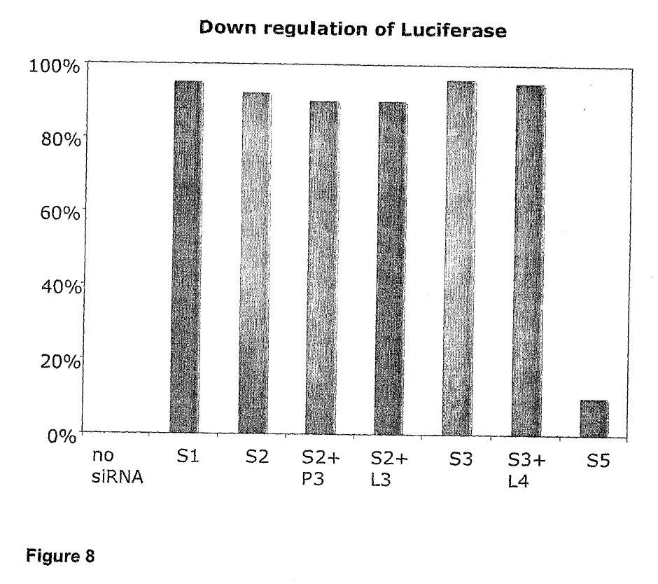

Down regulation (as a measure of siRNA efficacy) was measured by the luciferase assay (described earlier) of lysates from HepG2 cells, which were first transfected with a plasmid containing the gene for a GFP/luciferase fusion protein. In FIG. 8 it can be seen that the extended form of siRNA is less potent in comparison to the original siRNA. Both LNA and bis-PNA further decrease the gene silencing, marginally but still. siRNA LucG2 (S3) on the other hand is an improved version of the original non-mismatched siRNA (S1). Once again, the LNA anchor decreases its activity to some extent. The loss of efficacy caused by LNA can be compensated for by opening up of the 5′-end of the antisense strand.

### EXAMPLE 4

### Adding FE to siRNA Through LNA

Receptor mediated uptake of DNA by liver cells using carbohydrates, as functional entities is an attractive approach. Trimeric N-acetylgalactosamine (GalNAc) has a very high binding affinity to the asialoglycoprotein receptor, which is highly expressed on hepatic cells. Trimeric sugars were conjugated to a LNA anchor (L5) to enhance specific uptake of genetic material.

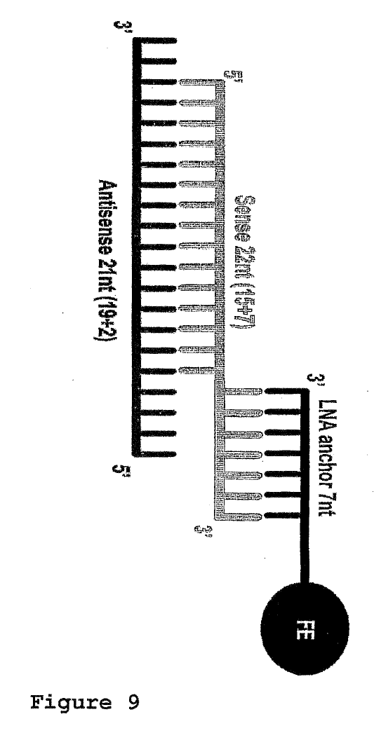

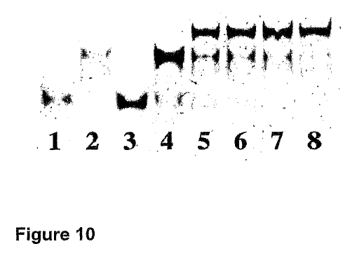

The synthesis of the trimeric carbohydrate unit is described in detail by Westlind et al., Glycoconj J. 2004;21(5):227-41. We choose to continue with the construct found to be most promising in the earlier experiments (see FIG. 9).

Choosing the 3′-end of the sense strand as anchor-binding domain might be advantageous, since there is already a 2 nucleotides extension in the general design. The sense strand's only purpose in an normal siRNA is thought to be for recruiting RISC to disassociate the strands and routing the anti-sense strand to the correct target mRNA. Studies have also shown that RISC preferentially attacks the weakest end of siRNA in terms of ΔG and binds to whichever strand that has its 5′-end open. When an anchor is hybridized as in FIG. 9, RISC would bind to the anti-sense strands 5′-end.

The siRNALucG2 (S3) was hybridized to an anchor, holding a trimeric carbohydrate as FE i.e. (L5) as described earlier. Lane 5-8 is a titration of L5. Already at equimolar ratio of L5 and S3 more than 90% of the siRNA is hybridized. Hence, the FE does not interfere significantly with the complex formation.

### EXAMPLE 5

### Gene Silencing Efficiency of Modified siRNA Hybridized to LNA with FE

HepG2 cells were transfected and treated as in Example 3 with LNA3S (L5) hybridized to siRNA LucG2 (S3)(described in Example 4). The Bioplex siRNAs gene silencing affect was compared with unmodified siRNA.

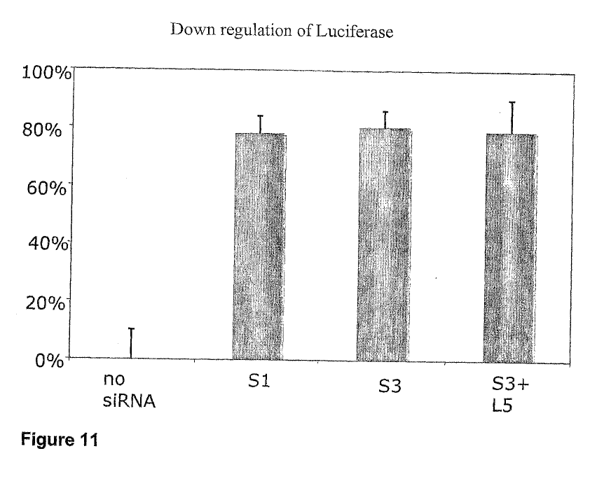

As can be seen in FIG. 11, modification of the siRNA and the addition of an anchor with a functional entity did not significantly effect the down regulation of Luciferase.

### EXAMPLE 6

### Effect of the FE Upon Uptake Into HepG2 Cells.

The functionality of siRNA is not quenched by anchors holding a FE, but is the FE (in this example carbohydrate) available for cellular interactions?

Hepatocytes are known to internalize shorter oligonucleotides through ODN cell membrane receptor. However, this pathway can be blocked by saturating the receptor with a 100-fold excess of an unrelated, short oligonucleotide (D5) (de Diesbach et al., Nucleic Acids Res. 2000 February 15;28(4):868-74).

150 000 HepG2 cells were grown for 24 hours in each well of a 24-well dish. Cells were washed with PBS and incubated in 150 μl serum free Optimem (Invitrogen) with 20 pmol of constructs and 2 nmol of decoy (D5) (in order to saturate the ODN membrane receptor) at 37° C. in a CO₂-incubator for 1 h. The cells were washed with PBS and trypsinized by the addition of 100 μL Trypsin (Invitrogen) to remove bound, but not internalized constructs. Finally the trypsin was removed by centrifugation and the cells were resuspended in PBS 1 mL supplemented with 3% fetal calf serum and kept on ice.

Constructs tested were; unhybridized siRNA (S3), S3 hybridized with L4 and L5 (LNA without and with carbohydrate moieties respectively). The siRNA was cy-5 labeled for detection by flow cytometric analysis (FACS). The cy-5 signal detected from untransfected cells was gated as negative, cells with higher intensity were considered as positive. 10 000 cells were measured for each construct and measured in a FACSCalibur (Becton Dickson, Franklin Lanes, N.J., USA) and analyzed with CellQuest software (Becton Dickson).

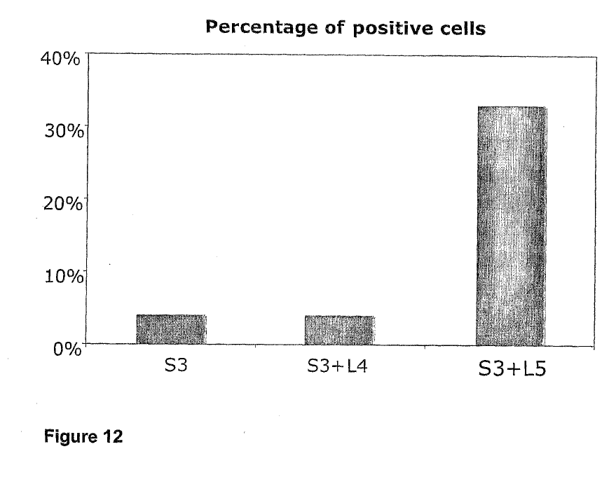

The FE conjugated to the LNA anchor enhanced cellular uptake of siRNA (S3+L5)dramatically in comparison to naked siRNA (S3)and siRNA hybridized to LNA (S3+L4) without carbohydrate (see FIG. 12). Percentage of positive cells relates to the number of liver cells that has taken up siRNA.

### EXAMPLE 7

### Verification of in Vivo Activity of siRNA LucG2 Hybridized to LNAG2

To investigate whether modified siRNAs also inhibit gene expression in vivo, we used the hydrodynamic transfection method. siRNA is delivered to the livers of adult mice through the tail vein. (Lewis D L, Wolff J A. Delivery of siRNA and siRNA expression constructs to adult mammals by hydrodynamic intravascular injection. Methods Enzymol. 2005;392:336-50.)

Mice were co-injected with one of the following; no siRNA, S3 or S3+L5, in combination with a luciferase-expression plasmid. All siRNA constructs were targeting the luciferase mRNA. Mice (30 g) were given 40 μg of siRNA construct and 10 μg of luciferase-expression plasmid in a volume of 2 ml Ringer's solution and with injection duration of 5-8 s. We monitored luciferase expression in living animals using quantitative whole body imaging (IVIS 100 system, Xenogen Corpration, Alameda, Calif., USA) 24 h after injections. Luciferin (Xenogen Corpration) was given by intraperitoneal route with a dose of 150 mg luciferin/kg body weight, in PBS. Animals were assayed 10 minutes post luciferin injection and exposure time was 3 seconds. During all experimental procedures the animals were anesthetized by continued administration of 2.5-3.5% isofluorane. All animal experiments were approved by the local ethical committee and conducted according to the Swedish guidelines.

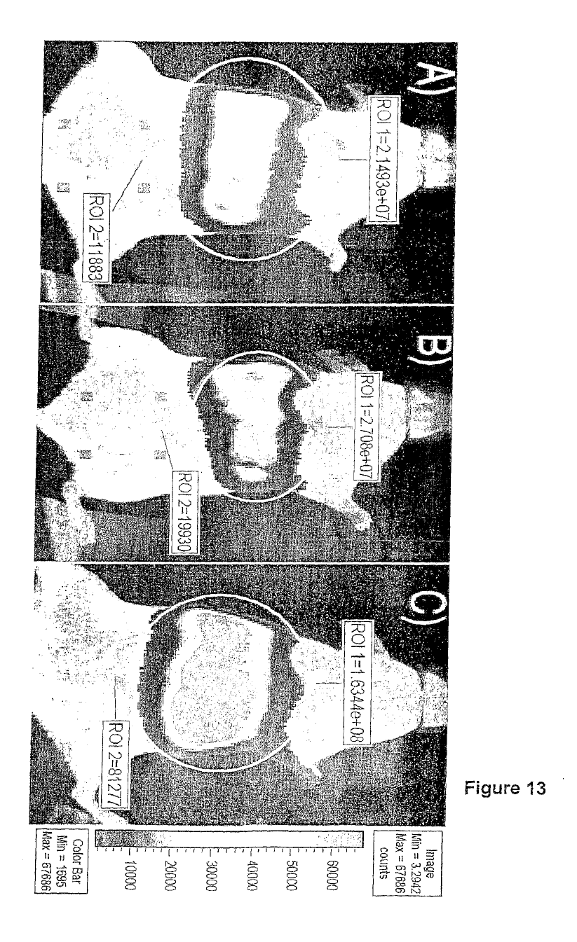

S3 or S3 hybridized to L5 was shown to exhibit equal potency on down regulation of the luciferase gene as can be seen in FIG. 13.

### EXAMPLE 8

### siRNA Induced Silencing at Transcriptional Level

From recent publications (Hong K, et al. Biomol Eng. 2001 October 31;18(4):185-92. Kawasaki H, et al. Nature. 2004 September 9;431(7005):211-217. Epub 2004 August 15. Jenke A C, et al. Mol Biol Rep. 2004 June; 31(2):85-90. Morris K V, et al. Science. 2004 August 27; 305(5688):1289-92. Epub 2004 Aug. 05) we have learned that siRNA can be used for silencing also at a transcriptional level. Transcriptional silencing in mammalian cells is associated with chromatin modifications that include histone deacetylation and cytosine DNA methylation. Treating cells with drugs reverse silencing by siRNA. Trichostatin (TSA) and 5-azacytidine (5-azaC) are inhibitors of histone deacetylases and DNA methyltransferases, respectively. These agents have been shown not affect RNA interference of the reporter gene transcript.

Since this is a nuclear event, NLS has been used for translocation of the siRNA. Non-covalent mix of siRNA and NLS-peptide showed increased silencing. The Bioplex technology would then have the advantage of a defined construct and increased complex stability in vivo (with respect to NLS siRNA complex formation). As mentioned in this application, we can also add other functions for cell internalization and specificity.

A new siRNA (S4) was purchased, targeting the same mRNA for luciferase but with an anchor-binding domain for PNA579 (P4). This bisPNA has a NLS peptide and will bring the siRNA into the nucleus where it is inactive. If we instead had chosen a siRNA targeting DNA within the nuclear chromatin, the NLS peptide facilitating nuclear uptake could potentially give a better silencing effect.

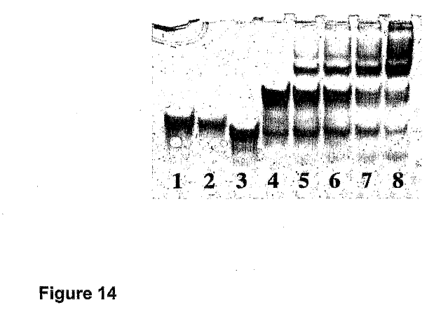

The protocols used are the same as described in materials and methods earlier. The results of the PAGE retardation assay are shown in FIG. 14. The siRNA LucGsite (S4) was hybridized to an anchor, holding the NLS peptide as FE, (P4) as described earlier. Lane 5-8 is a titration of L5. At a 1.2-fold excess of P4 to S4, more than 90% of the siRNA is hybridized. Hence, the FE does not interfere with the complex formation. The concentrations of constructs might not be exact, thus the excess could be a reflection of concentration measurements and pipetting.

“siRNA induced silencing at transcriptional level” is a negative proof of concept for NLS function. Transcriptional down regulation means that siRNA has to be introduced into the nucleus in order to bind to the DNA. The “normal” post translational down regulation takes place in the cytoplasm. RISC binds to siRNA in the cytoplasm and guides the antisense strand to the mRNA located in the cytoplasm.

Hence, we have mixed the two technologies by adding a NLS to a siRNA possessing post translational activity. To down regulate the expression of the Luciferase gene the siRNA has to be located in the cytoplasm where the RISC binds to the siRNA and locates the mRNA. On the contrary, the NLS peptide transfers the siRNA directly to the nucleus where no matching mRNA can be found.

### EXAMPLE 9

### Gene Silencing Capacity in the Nucleus.

Cos-7 cells were transfected and treated as in Example 3. The siRNA down regulation of luciferase protein was measured as described earlier. Normal siRNA (S1) was used as standard. The potency of modified siRNAs (S3 and S5, unhybridized or hybridized with L4, L5, S4 or S5), was measured as increase or decrease of down regulation in comparison to S1. Thus, the values obtained reflect relative increase or decrease in luciferase down regulation caused by siRNA and/or anchor and/or FE.

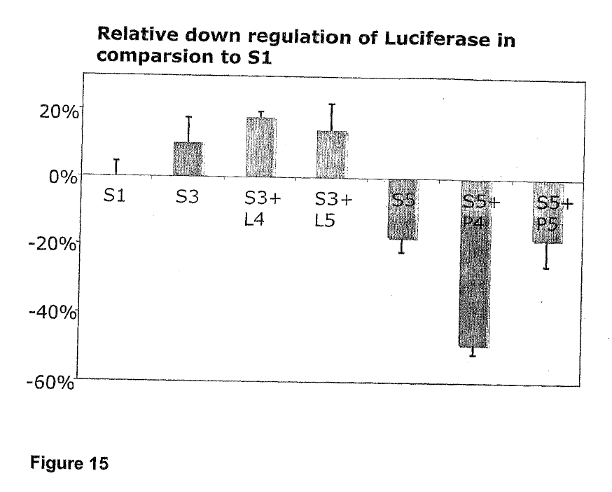

As can be seen in FIG. 15, adding NLS to a siRNA that induces silencing at the mRNA level, decreases the activity. S5+P5, which is a complex with the inverted NLS sequence results in a down regulation comparable with S5 without P5. Thus, the NLS is responsible for the loss of potency due to nuclear translocation of the siRNA complex.

The difference between S1 and S5 is the due to the modification, i.e. extension of the sense strand on the S5. The siRNA has not been tested with an optimal modification to present the anchor-binding domain but the effect of the NLS can clearly be seen as well as the importance of the siRNA design and the anchor-binding domain.

### EXAMPLE 10

### Intracellular Location of Complexes Having NLS

50 000 Cos-7 cells were grown for 24 hours in a 24-well plate. The cells were washed with PBS and incubated at 37° C. After overnight incubation, cells were transfected with 1 μg fluorescently labeled double stranded DNA (D6), mimicking the siRNA S5, using Lipofectamine (Invitrogen) according to the protocol for oligonucleotide transfection supplied by the manufacturer. The cells were incubated for 4 hours at 37° C. in 5% CO₂-incubator. The cells were washed with PBS and stained with DAPi nuclear dye (Invitrogen) according to the protocol supplied by the manufacturer and then washed with PBS and maintained in PBS.

D6 was hybridized to PNA conjugated to either the NLS peptide (P4) or the inverted (inactive) sequence (P5) and introduced to the cell culture. Live cells were analyzed by confocal microscopy (results not shown).

The siRNA complexes accumulate in the cell nucleus when they have a NLS peptide (S5+P4) attached. When the inverted NLS peptide is used (S5+P5), the signal is evenly distributed throughout the cell. The complex formation of siRNA with the NLS peptide does not significantly influence the NLS effect.

### EXAMPLE 11

### siRNA with Multiple FEs

The siRNALucGsite (S4) was hybridized to a bisPNA anchor (P6), having a linear stretch of PNA linked to it. A 33mer DNA oligonucleotide (D7) was hybridized to the linear PNA stretch of P6, at the5′ end of D7. Two anchor-binding domains for L5 are positioned at the 3′ end of D7.

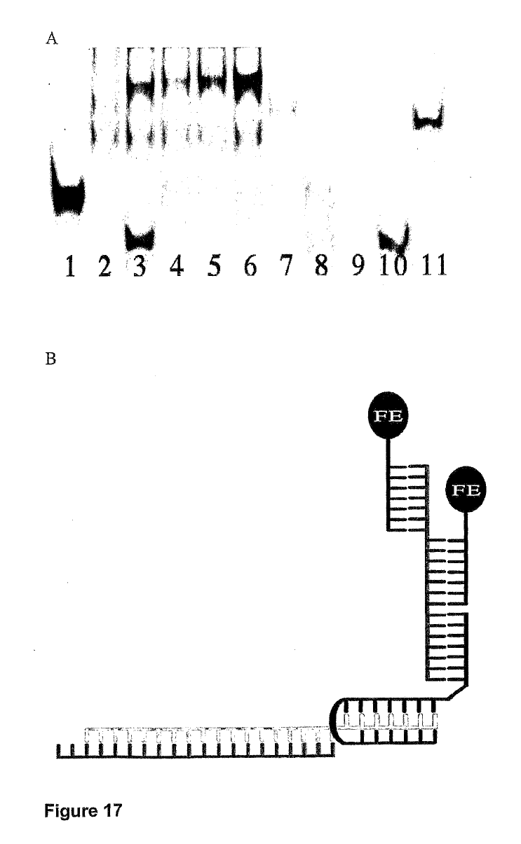

The complex was assembled under the conditions described earlier (37° C. for half an hour in Rignes buffer, 147 mM NaCl, 4 mM KCl₂ and 1.13 mM CaCl₂, pH 7.2). The order of the hybridization is indicated in FIG. 17. When consecutive hybridization was performed, the sample was incubated at 37° C. for half an hour per hybridization before the next construct was added. The building blocks of the complex were added as follows, 20 pmol S4, 24 pmol P6, 29 pmol D7 and 69 pmol L5. Final concentration of siRNA was 2 μM.

The complexes were run on PAGE shift assay (described earlier). The results are shown in FIG. 17A. The order of the hybridization is of no major importance. The siRNA complex could be assembled by simply adding all constructs simultaneously and incubated for 30 minutes at physiological conditions. In this experiment, excess were used, why unhybridized constructs can be seen in the gel. Lanes 4-6 are all comprising the entire complex, which is the least retarded band in the gel. In lane 9 only L5 is loaded and is poorly labeled by Sybr Green (Invitrogen), lane 3 was overloaded with 100 pmol of D7 by mistake.

### EXAMPLE 12

### Stability of siRNA with Anchor-FE

The siRNA LucG2 (S3)was hybridized with a LNA anchor having a trimeric carbohydrate as FE (L5), as described earlier.

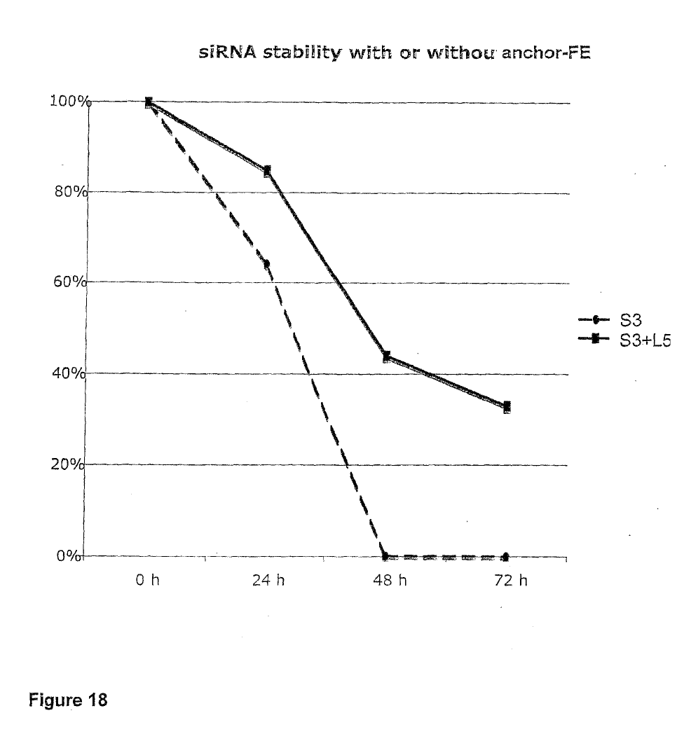

The complex (S3 or S3+L5)was incubated at a final concentration of 1 μM of siRNA, in Fetal Calf Serum (Invitrogen), at 37° C. At specified times, a 20 μL aliquot was removed, mixed with 40 μL formamide, flash frozen to −70° C., and stored at −20° C. Aliquots were taken at 0, 24, 48 and 72 hours and subsequently run on PAGE shift assay (described earlier). The band corresponding to the full complex was quantified and plotted in FIG. 18.

A resistance against degradation was observed for the S3+L5 complex (FIG. 18). The degradation could have been prevented even further by hybridizing a FE, such as, but not limited to, polyethylene glycol (PEG). Having PEG as FE on the siRNA is also a potential solution to avoid clearance. The size of the complex should then probably exceed 30 kDa.

Although particular embodiments have been disclosed herein in detail, this has been done by way of example for purposes of illustration only, and is not intended to be limiting with respect to the scope of the appended claims that follow. In particular, it is contemplated by the inventor that various substitutions, alterations, and modifications may be made to the invention without departing from the spirit and scope of the invention as defined by the claims.

1. A complex utilizing one or more functional entities (FEs), said FE(s) being capable of preventing degradation and/or removal of said complex, increasing the activity of said complex and/or increasing the transfer of said complex; extracellularly (within an organism), transcellularly (across a cellular membrane) and/or intracellularly (into different locations within a cell), characterized in that said complex comprises a short interfering RNA (siRNA) molecule, wherein said siRNA molecule comprises a first and a second strand and said siRNA molecule is modified in the following way:
at least one anchor-binding domain is incorporated into any one of the two strands of the siRNA molecule, said anchor-binding domain being a nucleic acid or analog thereof,
at least one anchor sequence is hybridized to said anchor-binding domain, said anchor sequence being a nucleic acid or analog thereof, and
at least one functional entity (FE) is linked to said at least one anchor sequence, said FE(s) being one or more biologically active molecule(s).

2. A complex according to claim 1, wherein the anchor-binding domain has one or more mismatches with respect to the other strand.

3. A complex according to claim 1, wherein the anchor-binding domain has 1-7 mismatches with respect to the other strand.

4. A complex according to claim 1, wherein the anchor-binding domain is a part of the siRNA molecule.

5. A complex according to claim 1, wherein the anchor-binding domain comprises up to 15 nucleotides.

6. A complex according to claim 1, wherein the anchor-binding domain comprises 3-12 nucleotides.

7. A complex according to claim 1, wherein the anchor-binding domain comprises 4-8 nucleotides.

8. A complex according to claim 1, wherein said anchor is a locked nucleic acid (LNA), peptide nucleic acid (PNA) or derivate thereof.

9. A complex according to claim 1, wherein the sense strand of the siRNA molecule acts as binding domain for the anchor sequence.

10. A complex according to claim 1, wherein the antisense strand of the siRNA molecule acts as binding domain for the anchor sequence.

11. A complex according to claim 1, wherein the siRNA molecule has a maximum length of 35 base pairs.

12. A complex according to claim 1, wherein the siRNA molecule has 3′ and/or 5′ overhangs of 1 to 12 nucleotides.

13. A complex according to claim 1, wherein the siRNA molecule has 3′ and/or5′ overhangs of 2 to 5 nucleotides.

14. A complex according to claim 1, wherein the siRNA molecule is chemically modified.

15. A complex according to claim 1, wherein the anchor-binding domain is an extension at either end of the sense or the antisense strand of the siRNA molecule.

16. A complex according to claim 1, wherein the complex comprises a cleavable linker molecule between said at least one FE and said at least one anchor sequence.

17. A complex according to claim 1, wherein the complex comprises a cleavable linker molecule between said at least one FE and said at least one anchor sequence and said linker comprises a disulphide bridge.

18. A complex according to claim 1, wherein the anchor sequence comprises a cleavable linker.

19. A complex according to claim 1, wherein the anchor sequence comprises a cleavable linker and said linker comprises a disulphide bridge.

20. A complex according to claim 1, wherein the anchor sequence is extended with at least one anchor-binding domain.

21. A method for transferring one or more functional entities (FEs) across a cellular membrane and into different locations within a cell wherein a complex according to claim 1 is used for transfection.

22. A method for making a complex that utilises one or more functional entities (FEs), said FE(s) being capable of preventing degradation and/or removal of said complex, increasing the activity of said complex and/or increasing the transfer of said complex; extracellularly (within an organism), transcellularly (across a cellular membrane) and/or intracellularly (into different locations within a cell), said complex comprises a short interfering RNA (siRNA) molecule and said siRNA molecule comprises a first and a second strand and wherein the method comprises the following steps:
introducing at least one anchor-binding domain into the siRNA molecule, said anchor-binding domain being a nucleic acid or analog thereof,
hybridizing at least one anchor sequence to said anchor-binding domain, said anchor sequence being a nucleic acid or analog thereof, and
linking at least one functional entity (FE) to said at least one anchor sequence, said FE(s) being one or more biologically active molecule(s).

23. A complex utilizing one or more functional entities (FEs), said FE(s) being capable of preventing degradation and/or removal of said complex, increasing the activity of said complex and/or increasing the transfer of said complex; extracellularly (within an organism), transcellularly (across a cellular membrane) and/or intracellularly (into different locations within a cell), characterized in that said complex comprises a short hairpin RNA (shRNA) molecule, wherein said shRNA molecule has a double stranded region and a hairpin loop and said shRNA molecule is modified in the following way:
at least one anchor-binding domain is incorporated into the shRNA molecule, said anchor-binding domain being a nucleic acid or analog thereof,
at least one anchor sequence is hybridized to said anchor-binding domain, said anchor sequence being a nucleic acid or analog thereof, and
at least one functional entity (FE) is linked to said at least one anchor sequence, said FE(s) being one or more biologically active molecule(s).

24. A complex according to claim 23, wherein the anchor-binding domain is a part of the shRNA molecule.

25. A complex according to claim 23, wherein the anchor-binding domain comprises up to 15 nucleotides.

26. A complex according to claim 23, wherein the anchor-binding domain comprises 3-12 nucleotides.

27. A complex according to claim 23, wherein the anchor-binding domain comprises 4-8 nucleotides.

28. A complex according to claim 23, wherein said anchor is a locked nucleic acid (LNA), peptide nucleic acid (PNA) or derivate thereof.

29. A complex according to claim 23, wherein the double stranded region of the shRNA molecule has a maximum length of 35 base pairs.

30. A complex according to claim 23, wherein the shRNA molecule has 3′ and/or5′ overhangs of 1 to 12 nucleotides.

31. A complex according to claim 23, wherein the shRNA molecule has 3′ and/or5′ overhangs of 2 to 5 nucleotides.

32. A complex according to claim 23, wherein the shRNA molecule is chemically modified.

33. A complex according to claim 23, wherein the anchor-binding domain is an extension at either end of the shRNA molecule.

34. A complex according to claim 23, wherein the anchor-binding domain is in the hairpin loop of the shRNA molecule.

35. A complex according to claim 23, wherein the anchor-binding domain is in one of the two strands of the double stranded region of the shRNA molecule.

36. A complex according to claim 23, wherein the anchor-binding domain is partly in the hairpin loop and partly in one of the two strands of the double stranded region of the shRNA molecule.

37. A complex according to claim 35, wherein the anchor binding domain has one or more mismatches with respect to the other strand of the double stranded region of the shRNA molecule.

38. A complex according to claim 35, wherein the anchor-binding domain has 1-7 mismatches with respect to the other strand of the double stranded region of the shRNA molecule.

39. A complex according to claim 23, wherein the complex comprises a cleavable linker molecule between said at least one FE and said at least one anchor sequence.

40. A complex according to claim 23, wherein the complex comprises a cleavable linker molecule between said at least one FE and said at least one anchor sequence and said linker comprises a disulphide bridge.

41. A complex according to claim 23, wherein the anchor sequence comprises a cleavable linker.

42. A complex according to claim 23, wherein the anchor sequence comprises a cleavable linker and said linker comprises a disulphide bridge.

43. A complex according to claim 23, wherein the anchor sequence is extended with at least one anchor-binding domain.

44. A method for transferring one or more functional entities (FEs) across a cellular membrane and into different locations within a cell wherein a complex according to claim 23 is used for transfection.

45. A method for making a complex that utilises functional entities (FEs), said FE(s) being capable of preventing degradation and/or removal of said complex, increasing the activity of said complex and/or increasing the transfer of said complex; extracellularly (within an organism), transcellularly (across a cellular membrane) and/or intracellularly (into different locations within a cell), said complex comprises a short hairpin RNA (shRNA) molecule and said shRNA molecule has a double stranded region and a hairpin loop, wherein the method comprises the following steps:
introducing at least one anchor-binding domain into the shRNA molecule, said anchor-binding domain being a nucleic acid or analog thereof,
hybridizing at least one anchor sequence to said anchor-binding domain, said anchor sequence being a nucleic acid or analog thereof, and
linking at least one functional entity (FE) to said at least one anchor sequence, said FE(s) being one or more biologically active molecule(s).

46. A complex utilizing one or more functional entities (FEs), said FE(s) being capable of preventing degradation and/or removal of said complex, increasing the activity of said complex and/or increasing the transfer or said complex; extracellularly (within an organism), transcellularly (across a cellular membrane) and/or intracellularly (into different locations within a cell), characterized in that said complex comprises an AS (antisense) molecule, wherein said AS molecule is modified in the following way:
at least one anchor-binding domain is incorporated or attached to the AS molecule, said anchor-binding domain being a nucleic acid or analog thereof,
at least one anchor sequence is hybridized to said anchor-binding domain, said anchor sequence being a nucleic acid or analog thereof, and
at least one functional entity (FE) is linked to said at least one anchor sequence, said FE(s) being one or more biologically active molecule(s).

47. A complex according to claim 46, wherein the anchor-binding domain is a part of the AS molecule.

48. A complex according to claim 46, wherein the anchor-binding domain comprises up to 15 nucleotides.

49. A complex according to claim 46, wherein the anchor-binding domain comprises 3-12 nucleotides.

50. A complex according to claim 46, wherein the anchor-binding domain comprises 4-8 nucleotides.

51. A complex according to claim 46, wherein said anchor is a locked nucleic acid (LNA), peptide nucleic acid (PNA) or derivate thereof.

52. A complex according to claim 46, wherein the antisense molecule has a maximum length of 35 bases.

53. A complex according to claim 46, wherein the AS molecule is chemically modified.

54. A complex according to claim 46, wherein the anchor-binding domain is an extension at either end of the strand of the AS molecule.

55. A complex according to claim 46, wherein the complex comprises a cleavable linker molecule between said at least one FE and said at least one anchor sequence.

56. A complex according to claim 46, wherein the complex comprises a cleavable linker molecule between said at least one FE and said at least one anchor sequence and said linker comprises a disulphide bridge.

57. A complex according to claim 46, wherein the anchor sequence comprises a cleavable linker.

58. A complex according to claim 46, wherein the anchor sequence comprises a cleavable linker and said linker comprises a disulphide bridge.

59. A complex according to claim 46, wherein the anchor sequence is extended with at least one anchor-binding domain.

60. A method for transferring one or more functional entities (FEs) across a cellular membrane and into different locations within a cell wherein a complex according to claim 46 is used for transfection.

61. A method for making a complex that utilises functional entities (FEs), said FE(s) capable of preventing degradation and/or removal of said complex, increasing the activity of said complex and/or increasing the transfer of said complex; extracellularly (within an organism), transcellularly (across a cellular membrane) and/or intracellularly (into different locations within a cell) and said complex comprises an AS (antisense) molecule, wherein the method comprises the following steps;
incorporating or attaching at least one anchor-binding domain to the AS molecule, said anchor-binding domain being a nucleic acid or analog thereof,
hybridizing at least one anchor sequence is to said anchor-binding domain, said anchor sequence being a nucleic acid or analog thereof, and
linking at least one functional entity (FE) to said at least one anchor sequence, said FE(s) being one or more biologically active molecule(s).

62. A cell transfected with the complex according to claim 1.

63. A kit comprising components for producing a complex capable of transferring one or more siRNA, shRNA or AS molecules across a cellular membrane (intracellularly, extracellularly and/or transcellularly) and into different locations within a cell, said kit comprising at least one siRNA, shRNA and/or AS molecule, at least one anchor-binding domain, at least one anchor sequence being able to hybridize to said anchor-binding domain, and at least one functional entity (FE) linked to said anchor-binding domain, said FE(s) being one or more biologically active molecule(s).


SEQUENCE LISTING (25 sequences)

SEQ ID NO: 1 - DNA, 5 bp, Artificial Sequence
  Description of Artificial Sequence Synthetic oligonucleotide
cccct

SEQ ID NO: 2 - DNA, 6 bp, Artificial Sequence
  Description of Artificial Sequence Synthetic oligonucleotide
cccctt

SEQ ID NO: 3 - DNA, 7 bp, Artificial Sequence
  Description of Artificial Sequence Synthetic oligonucleotide
ccccttt

SEQ ID NO: 4 - DNA, 7 bp, Artificial Sequence
  Description of Artificial Sequence Synthetic oligonucleotide
accgacc

SEQ ID NO: 5 - DNA, 21 bp, Artificial Sequence
  Description of Combined DNA/RNA Molecule Synthetic oligonucleotide
cuuacgcugaguacuucgatt

SEQ ID NO: 6 - DNA, 21 bp, Artificial Sequence
  Description of Combined DNA/RNA Molecule Synthetic oligonucleotide
ucgaaguacucagcguaagtt

SEQ ID NO: 7 - RNA, 26 nt, Artificial Sequence
  Description of Artificial Sequence Synthetic oligonucleotide
cuuacgcugaguacuucgaaaagggg

SEQ ID NO: 8 - DNA, 21 bp, Artificial Sequence
  Description of Combined DNA/RNA Molecule Synthetic oligonucleotide
ucgaaguacucagcguaagtt

SEQ ID NO: 9 - DNA, 24 bp, Artificial Sequence
  Description of Combined DNA/RNA Molecule Synthetic oligonucleotide
cuuacgcugaguacuaaggucggt

SEQ ID NO: 10 - DNA, 21 bp, Artificial Sequence
  Description of Combined DNA/RNA Molecule Synthetic oligonucleotide
ucgaaguacucagcguaagtt

SEQ ID NO: 11 - RNA, 25 nt, Artificial Sequence
  Description of Artificial Sequence Synthetic oligonucleotide
cuuacgcugaguacuaaaagaagaa

SEQ ID NO: 12 - DNA, 21 bp, Artificial Sequence
  Description of Combined DNA/RNA Molecule Synthetic oligonucleotide
ucgaaguacucagcguaagtt

SEQ ID NO: 13 - DNA, 24 bp, Artificial Sequence
  Description of Artificial Sequence Synthetic oligonucleotide
cttacgctgagtactaaggtcggt

SEQ ID NO: 14 - DNA, 18 bp, Artificial Sequence
  Description of Artificial Sequence Synthetic oligonucleotide
cttagtactctacgtaag

SEQ ID NO: 15 - DNA, 19 bp, Artificial Sequence
  Description of Artificial Sequence Synthetic oligonucleotide
ccttagtactctacgtaag

SEQ ID NO: 16 - DNA, 20 bp, Artificial Sequence
  Description of Artificial Sequence Synthetic oligonucleotide
accttagtactctacgtaag

SEQ ID NO: 17 - DNA, 15 bp, Artificial Sequence
  Description of Artificial Sequence Synthetic oligonucleotide
agatcagaccggatc

SEQ ID NO: 18 - RNA, 25 nt, Artificial Sequence
  Description of Artificial Sequence Synthetic oligonucleotide
cuuacgcugaguacuaaaagaagaa

SEQ ID NO: 19 - DNA, 21 bp, Artificial Sequence
  Description of Combined DNA/RNA Molecule Synthetic oligonucleotide
ucgaaguacucagcguaagtt

SEQ ID NO: 20 - DNA, 10 bp, Artificial Sequence
  Description of Artificial Sequence Synthetic oligonucleotide
ttcttctttt

SEQ ID NO: 21 - protein, 7 aa, Artificial Sequence
  Description of Artificial Sequence Synthetic peptide
PKKKRKV

SEQ ID NO: 22 - protein, 7 aa, Artificial Sequence
  Description of Artificial Sequence Synthetic peptide
VKRKKKP

SEQ ID NO: 23 - DNA, 15 bp, Artificial Sequence
  Description of Artificial Sequence Synthetic oligonucleotide
acatgcagtgttgat

SEQ ID NO: 24 - DNA, 33 bp, Artificial Sequence
  Description of Artificial Sequence Synthetic oligonucleotide
tgtacgtcacaactattggtcggtttggtcggt

SEQ ID NO: 25 - DNA, 10 bp, Artificial Sequence
  Description of Artificial Sequence Synthetic oligonucleotide
ttttcttctt